(19) United States
(12) Plant Patent Application Publication
(10) Pub. No.: US 2015/0237780 P1
(43) Pub. Date: Aug. 20, 2015

(54) Citrus rootstock named `UFR-6'

(50) ;

(71) Applicant: Florida Foundation Seed Producers, Inc., Marianna, FL (US)

(72) Inventor: Jude W. Grosser, Winter Haven, FL (US)

(21) Appl. No.: 14/544,570

(22) Filed: Jan. 22, 2015

Related U.S. Application Data
(60) Provisional application No. 61/942,575, filed on Feb. 20, 2014.

(51) Int. Cl. A01H 5/00

(52) U.S. Cl. PLT/201

(57) ABSTRACT

‘UFR-6’ is a new and distinct allotetraploid citrus rootstock for tree size control and improved disease resistance. ‘UFR-6’ has shown a positive reaction to the Huanglongbing disease (HLB, or citrus greening disease) in multiple experimental field trials. Scion trees grafted on this rootstock show a reduced frequency of infection and reduced disease symptoms once infected as compared to commercial diploid rootstocks.

### CROSS REFERENCE TO RELATED APPLICATIONS

This application claims the benefit of U.S. Provisional Application No. 61/942,575, filed Feb. 20, 2014, herein incorporated by reference in its entirety.

### BACKGROUND OF THE INVENTION

Latin name of the genus and species of the plant claimed: Citrus reticulata+Poncirus trifoliata

Variety denomination: ‘UFR-6’

The present invention relates to a new and distinct variety of citrus rootstock named ‘UFR-6’. The Plant Improvement Team in Lake Alfred, Fla. has pioneered the development and testing of allotetraploid citrus rootstocks. ‘UFR-6’ (identified as “Changsha+50-7” in field trials) is an allotetraploid somatic hybrid produced by protoplast fusion. Changsha mandarin is the embryogenic suspension culture parent and Argentine trifoliate orange is the leaf parent.

### BRIEF SUMMARY OF THE INVENTION

‘UFR-6’ was selected as a potential citrus rootstock on the basis of its reduced tree size, ability to produce sweet orange fruit with high solids, and positive reaction to Huanglongbing disease (HLB, or citrus greening disease) in multiple experimental field trials. Sweet orange scion trees grafted onto ‘UFR-6’ are precocious bearing, and trees generally do not exceed 10 feet in height when fully grown, making this rootstock a good candidate for use in Advanced Citrus Production Systems that feature high density plantings (Grosser et al., 2011). ‘UFR-6’ is seedy and highly polyembryonic, making it amenable to standard nursery propagation practices for uniform liner production. ‘UFR-6’ has also exhibited good cold hardiness. Long-term performance of trees on this rootstock selection is unknown. Trees on ‘UFR-6’ are susceptible to citrus blight, but its blight tolerance appears to be better than Swingle citrumelo, the most popular commercial citrus rootstock in Florida. True-to-typeness of UFR-6 through asexual propagation was demonstrated by planting cuttings from the original tree and a tree grafted to a somatic hybrid rootstock (Hamlin+rough lemon) in Osceola county, Florida. Seeds from this tree and the original tree are predominantly of nucellar origin and produce true-to-type seedlings.

### BRIEF DESCRIPTION OF THE DRAWINGS

‘UFR-6’ is illustrated by the accompanying photographs, which show the tree's form, foliage, and fruit. The colors shown are as true as can be reasonably obtained by conventional photographic procedures. The photographs are of a tree approximately 8 years old. All figures were taken in December of 2013.

FIG. 1.—Shows a close-up of the nearly mature fruits with the rind and cross-sectional view of the fruit when cut in the center.

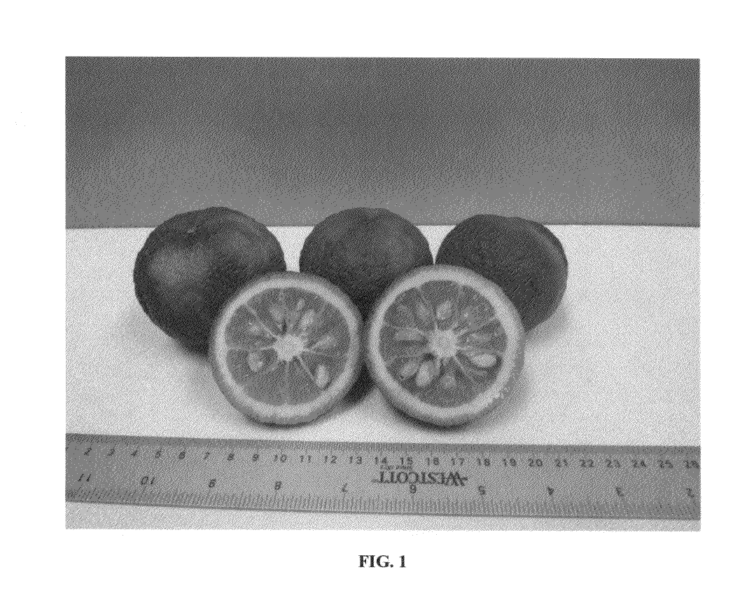

FIG. 2.—Shows the overall mature plant growth habit.

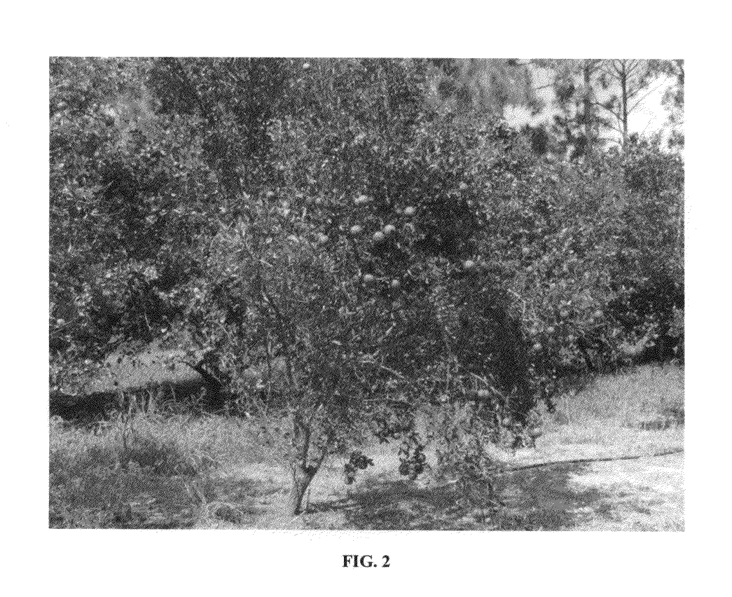

FIG. 3.—Shows nearly mature fruits hanging on the tree.

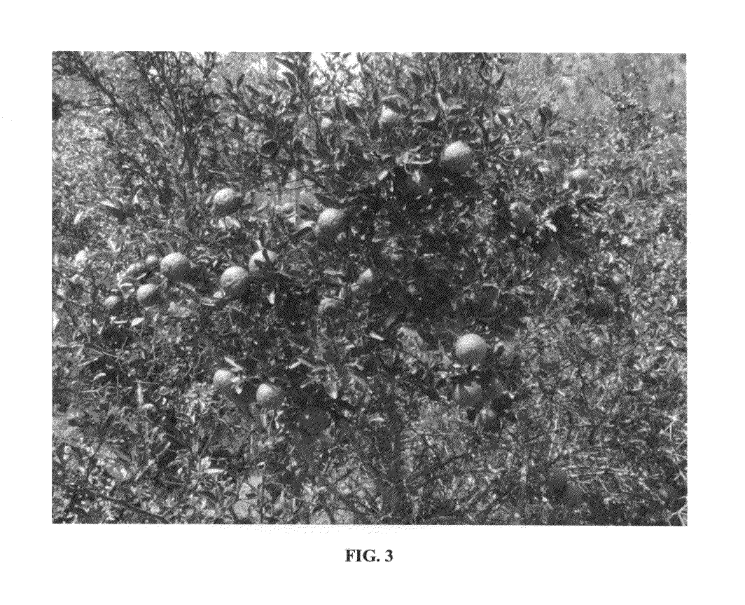

FIG. 4.—Shows a close-up of leaves and nearly mature fruits.

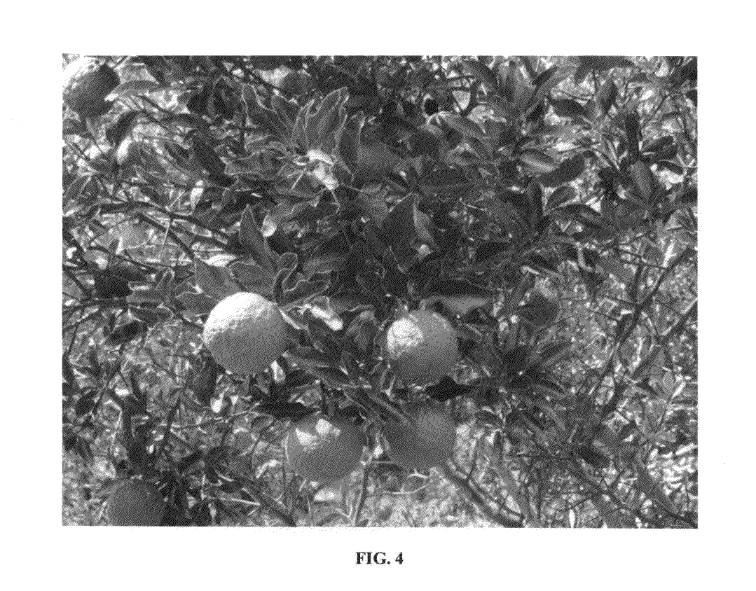

FIG. 5.—Shows a close-up of nearly mature fruits.

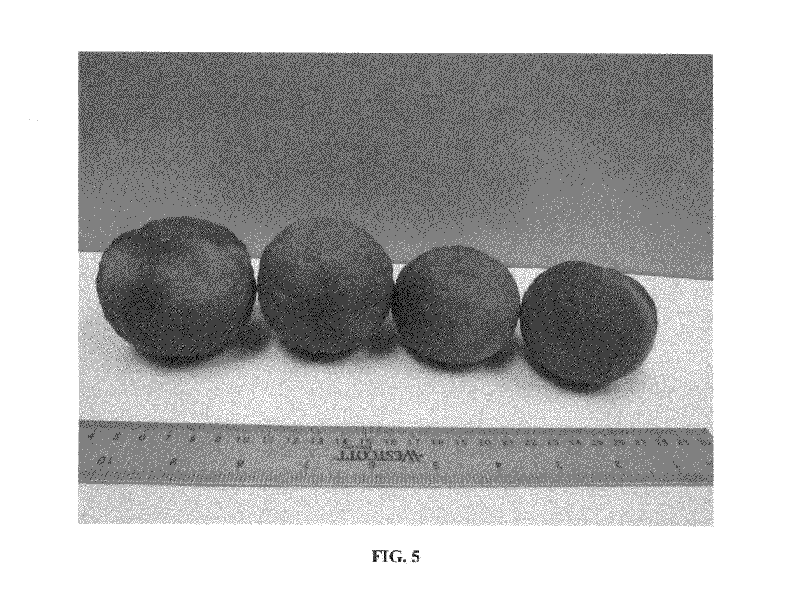

FIG. 6.—Shows a close-up of seeds from nearly mature fruit.

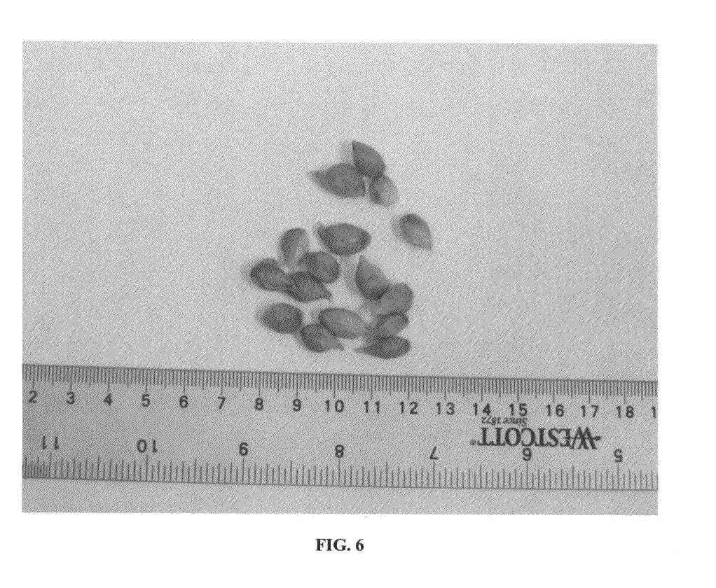

### DETAILED BOTANICAL DESCRIPTION

The following detailed description sets forth the distinctive characteristics of ‘UFR-6’. The colors (except those in common terms) are described from the R.H.S. Colour Chart published by The Royal Horticultural Society in London (second edition), in association with the Flower Council of Holland.
Phenotypic description of Citrus reticulata+Poncirus trifoliata ‘UFR-6’:
Classification:
   Botanical.—Citrus reticulata+Trifoliate orange Poncirus trifoliate.
   Common name.—Allotetraploid mandarin trifoliate orange somatic hybrid.
Parentage:
   Embryogenic culture parent.—‘Changsha mandarin’ (unpatented).
   Leaf parent.—‘50-7 trifoliate orange’ (unpatented).
Tree:
   Ploidy.—Tetraploid.
   Size.—Small.
   Height.—3.3 meters.
   Tree spread.—2.6 to 2.9 meters.
   Vigor.—Moderately vigorous.
   Density.—Canopies are moderately dense.
   Form.—The tree is obloid-shaped with lateral and upright branches growing.
   Growth habit.—Both upright and lateral growth with a low medium angle.
Trunk:
   Trunk diameter.—10.9 cm in diameter at 30 cm above the ground on a 16-year-old tree.
   Trunk texture.—Rough.
   Trunk bark color.—RHS 197A (greyed-green); irregularly striated with RHS N189A (green).
Branches:
   Crotch angle.—First crotch forms a 45- to 50-degree angle; middle crotch forms a 65-degree angle.
   Branch length.—Branch reaches 2.8 meters from the first crotch to the tip of the branch.
   Branch texture.—Relatively rough with small thorns or spines.
   Branch color (shoots from previous flush, hardened and 4 to 5 mm in diameter).—RHS N137A (green).
Leaves:
   Size (lamina average).—Length: 64.5 mm Width: 39.1 mm L/W ratio: 1.65.
   Thickness.—Thicker than average diploid citrus rootstock hybrids.
   Type.—Mostly simple leaf types, with trifoliate types of leaves appearing within the same tree.
   Shape.—Elliptical.
   Apex.—Retuse.
   Base.—Acute to sub-obtuse.
   Margin.—Entire and irregular margin, slightly undulate.
   Surface.—Upper surface: Glabrous Lower surface: Medium veins that are pinnately netted.
   Color.—Upper surface (adaxial): RHS N137C (green) Lower surface (abaxial): RHS 138A (green).
   Petiole.—Shape: Brevipetiolate (shorter than leaf lamina); junction between petiole and lamina is articulate Width (petiole wing): Narrow Shape (petiole wing): Obovate Length: 16.1 to 18.1 mm Width: 4.4 to 5.3 mm Color: RHS N137C (green).
Flowers and flower buds:
   Type.—Hermaphrodite.
   Bearing.—Flowers grow from leaf axillaries and leaf terminals singly and in small clusters; most single flowers grow from leaf axillaries.
   Flower bud size.—Shape: Initial visible flower bud has a round ball shape; mature flower bud has an elongated olive shape.
   Flower petals.—Shape: Flat, spatula-shaped Apex shape: Smooth, acute-shaped Base shape: Even obtuse Margin: Smooth.
   Flower sepal.—Shape: Delta-shaped with an acute angle at the apex Apex shape: Triangle-shaped Margin: Smooth.
   Fragrance.—Fragrant/Moderately fragrant.
   Reproductive organs.—Fertility: Appears self-fertile Pollen amount: Abundant/Moderate amount Pollen color (general): Bright-yellow Ovary shape: Oval-shaped.
Fruit:
   Size.—Uniform.
   Height.—56.5 to 61 mm on average.
   Width.—63.8 to 71.2 mm on average.
   Average weight (per individual fruit).—147 grams.
   Shape.—Round.
   Shape (cross-section).—Round.
   Apex.—Truncated with slight dent.
   Apex cavity diameter.—N/A.
   Base cavity diameter.—5.0 to 6.5 mm.
   Base.—No neck.
   Harvesting.—Fruit can be harvested from October through December in Florida.
   Fruit stem (short stem connecting the fruit).—Length: 5.8 mm Diameter: 3.4 mm Color: RHS 146A (yellow-green) with RHS 195B (grey-green) strip.
Rind:
   Adherence.—Adherence between albedo (mesocarp) and flesh (endocarp) is strong. The adherence is evenly distributed from base to apex.
   Thickness.—7.3 to 7.5 mm on average.
   Texture.—Rough surface.
   Color.—Flavedo (epicarp): Ranges between RHS N144A (yellow-green) to RHS 146A (yellow-green) Albedo (mesocarp): RHS N155B (white).
   Stylar end.—Closed.
   Rind oil cell density.—246 oil cells/square cm.
Flesh:
   Number of segments.—Between 8 and 9 segments per fruit on average.
   Segment walls.—Firm with sufficient strength to maintain integrity as separated.
   Juice.—Abundant.
   Color.—Uniformly RHS 16C (yellow-orange).
   Texture.—Soft.
   Vesicles.—Length: Arranged from 8.7 to 9.9 mm on average Diameter (thickness): 3.0 to 3.8 mm on average.
   Eating quality.—N/A.
   Juice index.—
   Soluble solids (average).—8.5 Brix.
Seeds:
   Type.—Polyembryonic/Monoembryonic.
   Number.—Ranges from 30 to 37.
   Shape.—Seed shapes are not uniform. Normal seeds are mostly ventricose/swollen-shaped and clavate club shaped.
   Size.—Length: 15.9 to 16.4 mm Width: 7.2 to 9.8 mm.
   Seed coat color.—Outer Surface: RHS 156D (greyed-white) and wrinkled Inner surface: RHS 165C (greyed-orange) Cotyledon color: RHS 145A (yellow-green).
Resistance to disease: ‘UFR-6’ rootstock was selected on the basis of its positive reaction to HLB disease (huanglongbing or citrus greening disease) in multiple field trials. Trees on this rootstock show a reduced frequency of infection and reduced disease symptoms once infected as compared to commercial diploid rootstocks.

What is claimed is:

1. A new and distinct citrus rootstock cultivar as illustrated and described herein.
